(19) United States
(12) Patent Application Publication
(10) Pub. No.: US 2021/0195407 A1
(43) Pub. Date: Jun. 24, 2021

(54) SYSTEMS AND METHODS FOR WIRELESSLY PAIRING MEDICAL DEVICES USING NON-NUMERIC KEY PATTERNS

(71) Applicant: GE Precision Healthcare LLC, Wauwatosa, WI (US)

(72) Inventor: Cibu Jose, Waukesha, WI (US)

(73) Assignee: GE Precision Healthcare LLC, Wauwatosa, WI (US)

(21) Appl. No.: 16/720,975

(22) Filed: Dec. 19, 2019

(51) Int. Cl. H04W 12/00; H04W 76/10; G06F 3/0487; G06F 3/033; H04W 12/06; H04W 12/04; G16H 40/63

(52) U.S. Cl. CPC H04W 12/003; H04W 76/10; G06F 3/0487; G16H 40/63; H04W 12/06; H04W 12/0401; G06F 3/033

(57) ABSTRACT

A method for wirelessly pairing a medical acquisition device and a host device. The method includes requesting an authentication key associated with the medical acquisition device to be entered into the host device, and producing a key pattern on the medical acquisition device representative of the authentication key, where the key pattern is non-numeric. The method further includes providing a plurality of targets on the host device, and receiving selections of the plurality of targets to form an entry pattern on the host device. The method further includes wirelessly pairing the medical acquisition device and the host device only when the entry pattern is determined to match the key pattern on the medical acquisition device.

### FIELD

The present disclosure generally relates to systems and methods for wirelessly pairing medical devices using non-numeric key patterns, and more particularly to systems and methods for wirelessly pairing medical devices using non-numeric key patterns, particularly where one of the medical devices has four or more LEDS and no numeric display.

### BACKGROUND

Wireless, activity between devices is becoming more and more prevalent, including within the medical devices context in which wires become tangled and problematic, as well as limiting the mobility of a patient and/or medical devices. One mechanism or protocol for providing wireless connectivity between devices that has been widely accepted is Bluetooth®, which provide pairing between devices by successfully entering a six digit authentication key (each comprised of a number 0-9) corresponding to one device into a second device, whereby a matching entry is required for the two devices to successfully pair.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

One embodiment of the present disclosure generally relates to a method for wirelessly pairing a medical acquisition device and a host device. The method includes requesting an authentication key associated with the medical acquisition device to be entered into the host device, and producing a key pattern on the medical acquisition device representative of the authentication key. In this embodiment, the key pattern is non-numeric. The method further includes providing a plurality of targets on the host device, and receiving selections of the plurality of targets to form an entry pattern on the host device. The method further includes wirelessly pairing the medical acquisition device and the host device only when the entry pattern is determined to match the key pattern on the medical acquisition device.

Another embodiment generally relates to a method for wirelessly pairing a medical acquisition device and a host device within a time limit. The method includes providing a plurality of targets on the host device and requesting the medical acquisition device to sequentially produce six key patterns, each of the six key patterns being non-numeric and made of selections among a plurality of individual indicators. The method further includes sequentially receiving on the host device, one after each of the six key patterns is produced on the medical acquisition device, six entry patterns each comprised of selections among the plurality of targets. The method further includes indicating how many of the six entry patterns have been received by the host device, and indicating a remaining time until the time limit is reached. The method further includes wirelessly pairing the medical acquisition device and the host device only when the sequence of six entry patterns is determined to match the sequence of six key patterns on the medical acquisition device before the time limit is reached.

Another embodiment generally relates to a system for wirelessly pairing a medical acquisition device and a host device. A plurality of LEDs is operatively coupled to the medical acquisition device, where each of the plurality of LEDs is selectively illuminated to collectively form at least six key patterns, and where the at least six key patterns are non-numeric. A display device is operatively coupled to the host device, where the display device displays a plurality of targets thereon, and where each of the plurality of targets is selectable. An input device is operatively coupled to the host device, where the input device is operable to select among the plurality of targets to collectively form entry patterns. A computer system compares the at least six key patterns to the entry patterns corresponding thereto. The medical acquisition device and the host device wireless pair only when the at least six key patterns are determined to match the entry patterns corresponding thereto.

Various other features, objects and advantages of the disclosure will be made apparent from the following description taken together with the drawings.

### BRIEF DESCRIPTION OF THE DRAWINGS

The present disclosure is described with reference to the following Figures.

FIG. 1 depicts a front view of an exemplary medical acquisition device and host device wirelessly paired according to the present disclosure;

FIG. 2A is front view of an exemplary interface for the embodiment of medical acquisition device shown in FIG. 1;

FIG. 2B provides a listing of key patterns enterable from an interface of an alternative embodiment of medical acquisition device configured for pairing according to the present disclosure;

FIG. 3 is a front view of the medical acquisition device and host device of FIG. 1, now shown in the process of pairing according to the present disclosure;

FIG. 4 depicts an exemplary process flow for pairing a medical device and host device according to the present disclosure, such as may be followed for the pairing shown in process in FIG. 3; and

FIG. 5 depicts an exemplary control system as may be incorporated within the medical acquisition device and/or host device according to the present disclosure.

### DETAILED DISCLOSURE

The present disclosure generally relates to systems and methods for authenticating Bluetooth® or like wireless pairing protocols between two devices, such as a medical acquisition device and a host device. In particular, the present disclosure relates to authentication methods in which the input capabilities of one of the devices, such as the medical acquisition device, are limited. Bluetooth® is one such example of a wireless pairing protocol and has been widely accepted across many industries.

Security for Bluetooth® devices is tied to the pairing method employed. Bluetooth® provides for a wide range of security profiles, starting with no security, to authenticated pairing with Man In The Middle (MITM) protection. For medical devices, it is desired (if not required) to have the highest level of security that is possible, while also minimizing the cost and design constraints in the product or products being paired. The passkey entry pairing method of Bluetooth® is a pairing method that provides authenticated pairing with MITM protection. This method requires one device with display capability, and another device with key entry capability, to accomplish pairing. The device with the display capability must be capable of displaying a six digit pairing key, and likewise the other device with the key entry capability must be capable of entering these same six digit pairing keys.

However, the inventor has identified that many devices in which secure pairing is highly desirable are not suited for the pairing methods presently known. In the example of an ECG acquisition device, such as GE Healthcare's CAM CONNECT 14, the only indicators on the device are LEDs (in certain examples, tri-colored LEDS) provided to indicate the status of each ECG electrode connected thereto. For example, the LEDs may indicate that the ECG electrode is successfully connected, is disconnected, or has some error condition associated therewith.

Another challenge that is faced by users is the pairing timeout that is mandated by the Bluetooth® specification (referred to as the SMP Timeout). This timeout is implemented for security reasons to prevent the pairing protocol from stalling due to the lack of user input. The timeout clock starts from the instant when the initiating device sends the pairing request or the responding device receives the pairing request. If the pairing process is not completed within 30 seconds of the start of the timer, the pairing fails and the user has to re-initiate pairing.

The inventor has recognized that most users are not even aware of this timeout, or its value as mandated by the Bluetooth® specification. If a user is slow to enter the six digit key and the pairing timeout expires, resulting in failed pairing, users are often left wondering what caused the failure.

As will become apparent, the present systems and methods provide for Bluetooth® passkey entry pairing between devices that do not have the sufficient capability to display a six digit number, and are therefore unable to pair with a host device according to systems and methods presently known in the art. In particular, the inventor has recognized that other indicators provided on such a device, which are used for some other operation of the device rather than in a pairing process, may be repurposed to serve as representations of numeric numbers, and therefore may be used to provide pairing between these existing devices and a host device using the Bluetooth® protocol, non-numerically.

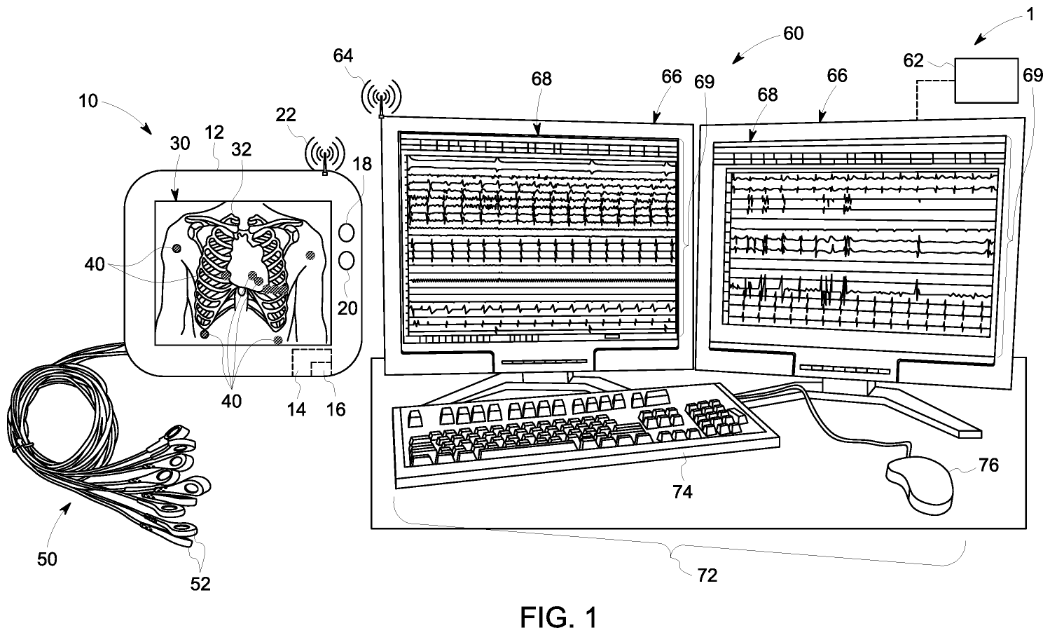

FIG. 1 depicts one such system 1 for pairing a medical acquisition device 10 with a host device 60 according to the present disclosure. In the present example, the medical acquisition device 10 is an ECG device having a main unit 12 and leads 50, which receive data from a patient by connecting the electrode couplers 52 of the leads 50 to electrodes positioned on a patient in the manner known in the art. The main unit 12 comprises a control system 14 for providing the functions of the ECG device, as well as for providing a pairing process to be discussed below. This includes an authentication key generator 16, which is provided within or operatively in conjunction with the control system 14. A main unit 12 further includes a pairing button 18 that initiates that pairing process between the medical acquisition device 10 and the host device 60, as well as an operating button 20 for controlling the standard operation of the ECG device in acquiring data from the patient. It should be recognized that the other buttons or forms of selection are also anticipated by the present disclosure.

The main unit 12 further includes a communication device 22, which in the present example is used for wirelessly communicating between the medical acquisition device 10 and the host device 60, particularly with a communication device 64 associated therewith.

As shown in FIG. 1, the medical acquisition device 10 in the present example does not include a digital display or alphanumeric indicator. However, the medical acquisition device 10 does have an indicator interface 30 that provides individual indicators 40 positioned on an anatomical graphic 32, which correspond to positions for electrode placement for obtaining ECG signals. In certain examples of medical acquisition devices 10 presently known in the art, these individual indicators 40 are tri-color status LEDs that each indicate an ECG signal quality for one of the leads 50, with ten individual indicators 40 being provided as specific indicators 11410. As will be discussed below, the inventor has identified that although these individual indicators 40 are presently used only for the standard operation of an ECG device, the fact that there are ten individual indicators 40 may be exploited such that each of the individual indicators 40 is assigned to one numeric digit 0-9. In this manner, the individual indicators 40 may be repurposed during the pairing process to represent an authentication key for medical acquisition device 10 that is otherwise not equipped to do so.

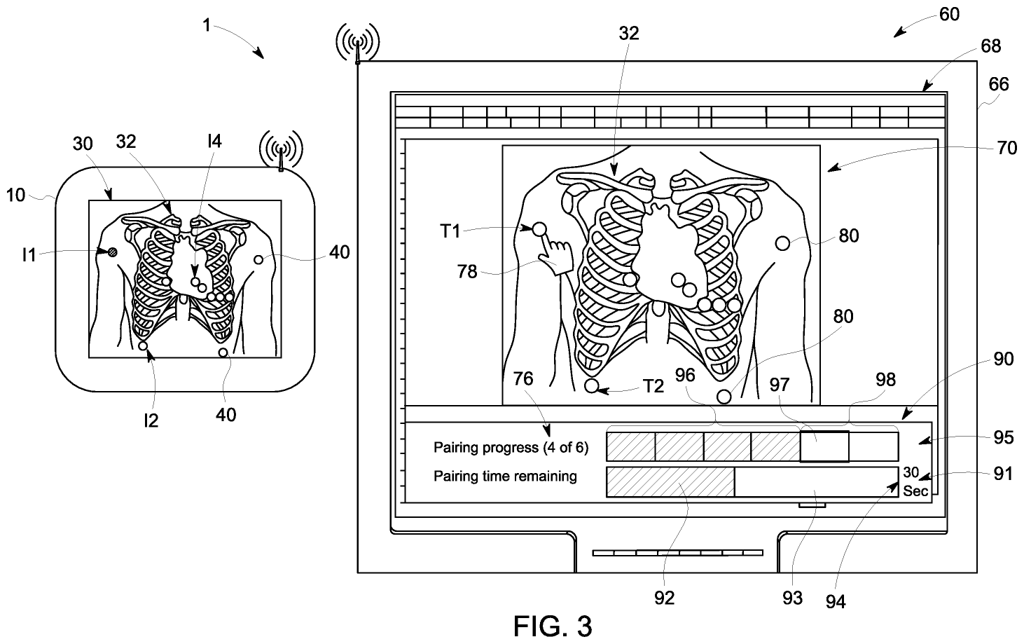

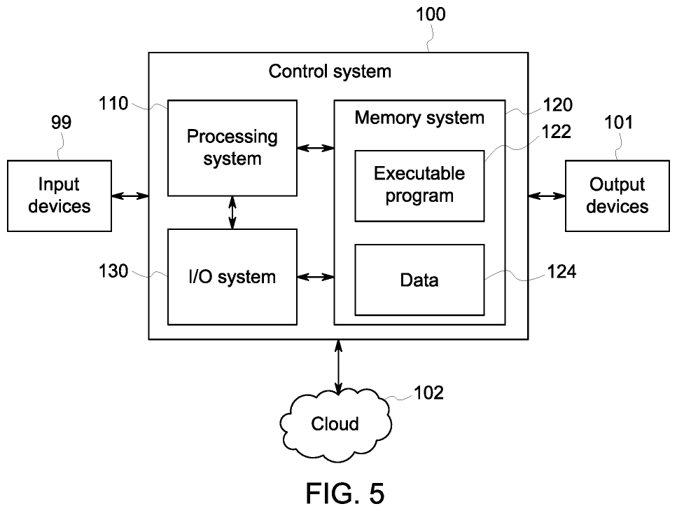

FIG. 1 further depicts an exemplary host device 60 shown receiving information from a medical acquisition device 10 wirelessly paired therewith, particularly received via the communication device 64 discussed above. The host device 60 includes a control system 62, which like control system 14 of the medical acquisition device 10 may be configured in the manner shown for the control system 100 of FIG. 5 (discussed further below). The host device 60 includes a display device 66, which in the present example is two computer monitors each displaying an interface 68 and displaying medical data 69. User input devices 72 are also operatively connected to the host device 60, which in the present example include a keyboard 74 and a mouse 76, which as will be discussed below provides for movement and selection via a curser 78 (see FIG. 3).

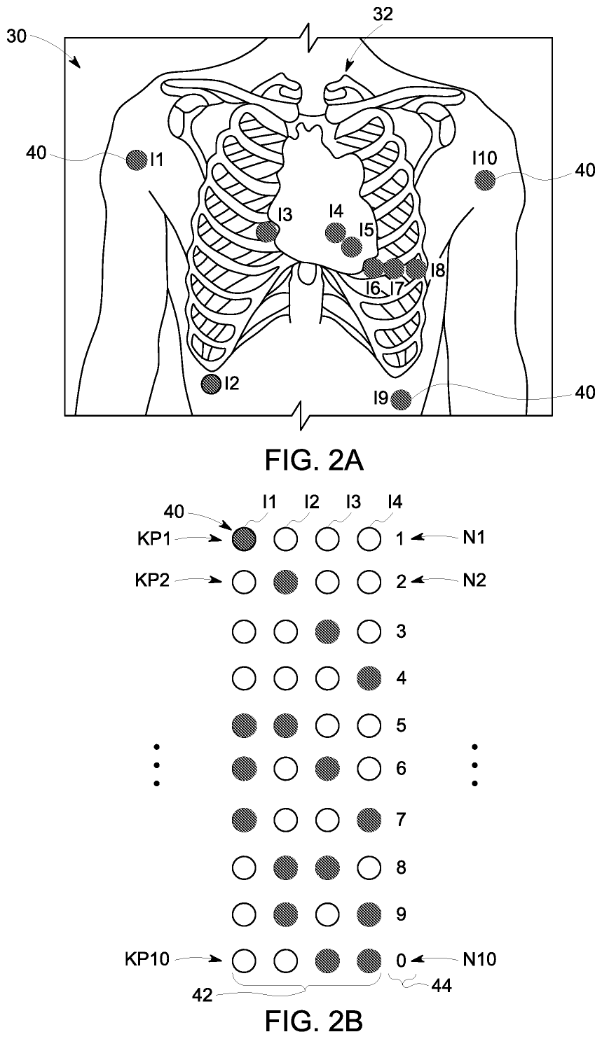

FIG. 2A further depicts the indicator interface 30 from the medical acquisition device 10 shown in FIG. 1, but now with each of the ten individual indicators 40 being assigned to a specific indicator I1-I10, respectively. In the example shown, each of the specific indicators I1-I10 is shown darkened, which might represent a LED being on or illuminated. Having all ten individual indicators 40 darkened in certain embodiments serves as an indication that the pairing process is starting, or that an entry has been received by the host device 60 and that a new pattern will then be displayed, for example. The particular configuration of which specific indicators I1-I10 are selected (e.g. illuminated) is also referred to herein as a key pattern 42, whereby at least one of the specific indicators I1 410 is illuminated in each key pattern 42. For example, a first specific key pattern KP1 may correspond to only the first of the specific indicators I1 being selected, a second specific key pattern KP2 distinct from the first when only the second of the specific indicators 12 is selected, and the like.

As shown in FIG. 2B, it is further possible that key patterns 42 may be provided whereby more than one specific individual indicator I1-I10 is illuminated, allowing all ten digits 0-9 to be represented with fewer than ten individual indicators 40. In the example shown, four individual indicators 40 are used, whereby ten key patterns 42 (listed as specific key patterns KP1-KP10) are each assigned to or represent a unique numeric equivalent 44 corresponding to the authentication key, referred to as specific numeric equivalents N1-N10. On this basis, it will be recognized that medical acquisition devices 10 (or other devices, medical or not) may be used to generate key patterns representative of digits in an authentication key with as few as four individual indicators 40 being present therewith.

In further examples, all ten key patterns 42 may be provided via a single individual indicator 40, provided it can indicate a sufficient number of distinct indications. For example, a device capable of displaying at least ten unique colors could be used and mapped to digits as follows: Light blue—0, Dark blue—1, Light Green—2, Dark Green—3, Red—4, Orange—5, Yellow—6, Purple—7, White—8, and Pink—9.

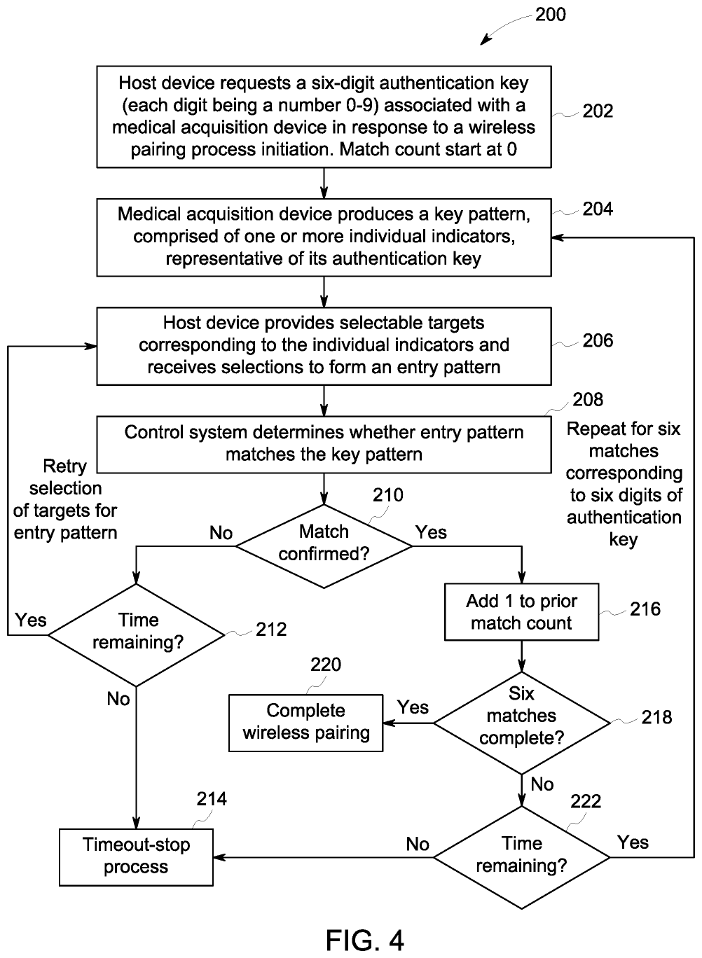

FIG. 3 depicts a medical acquisition device 10 and host device 60 now in the process of wirelessly pairing, rather than transmitting ECG data as shown in FIG. 1. In the present example, only the first specific indicator I1 is shown to be selected on the indication interface 30 of the medical acquisition device 10, whereby all other individual indicators 40 remain unselected. As will be discussed further below with respect to the process flow in FIG. 4, this indicator interface 30 may therefore be configured to correspond to a key pattern 42 having a numeric 44 of one. However, it is not necessary that the user is aware of the particular assignment of key patterns 42 to numeric equivalence 44. Instead, as shown on the interface 68 of the host device 60, the user merely identifies which of the individual indicators 40 is selected on the medical acquisition device 10, and subsequently selects the corresponding target 80 on a pairing display 70 shown on the display device 66 during the pairing process.

It will be recognized that the targets 80 correspond to the individual indicators 40 on the medical acquisition device 10 such that there are ten specific targets T1-T10 corresponding to the ten specific indicators I1-I10. The user then selects the one or more targets 80 in the pairing display 70 that corresponds to those selected on the medical acquisition device 10, constituting an entry pattern 82 that should match the key pattern 42 of the medical acquisition device 10. As discussed above, this may entail selecting more than one target 80, depending upon which of the individual indicators 40 on the medical acquisition device 10 are selected. The control system 62 (see FIG. 1) then compares whether the entry pattern 82 provided by the user on the pairing display 70 matches the key pattern 42 provided on the medical acquisition device 10, which allows the pairing process to proceed when properly matched. As will be discussed further below, this would be repeated until all digits have been properly entered into the host device 60, which for a standard Bluetooth® protocol would be 6 digits total.

The embodiment of FIG. 3 further depicts a progress display 90 that shows the progress of the pairing process in real-time, which is not presently provided with Bluetooth® pairing processes known in the art. In particular, Bluetooth® pairing processes are limited to thirty seconds maximum for entering all digits corresponding to the authentication key to complete the wireless pairing. However, users today are not aware of how much time is remaining, and therefore are subject to time outs and having to start the wireless pairing process over.

In the exemplary progress display 90 shown, a pairing time indicator 91 is provided, as well as a pairing progress indicator 95. The pairing time indicator 91 in the present example is a running bar depicting the amount of pairing time elapsed 92 relative to the pairing time remaining 93 and the overall pairing time limit 94, which is as previously discussed is generally thirty seconds. It will be recognized that once the initiation of the pairing process has started, for example via the pair button 18 on the medical acquisition device 10, the pairing time elapsed 92 runs from left to right until thirty seconds have elapsed and the process times out, unless pairing has completed before this time. This provides a much needed indication for the operator as to the pace for entering entry patterns 82 into the host device 60.

The progress display 90 further includes a pairing progress indicator 95, which shows the pairing matches already complete 96, which pairing match (for example of the six total required) is in progress 97, and how many pairing matches are remaining 98 before the pairing process is completed. By providing this pairing process indicator 95, the user may compare this status to the pairing time indicator 91 to identify the portion of the overall pairing process remaining to be completed.

FIG. 4 depicts an exemplary process flow 200 for pairing a medical acquisition device 10 (or other wireless device) with a host device 60, such as may be followed in the pairing shown in FIG. 3. The process begins in step 202 with the host device 60 requesting a six digit authentication key, whereby each digit is assigned a number 0-9, and whereby the authentication key is associated with a medical acquisition device 10. As discussed above, this request happens in the response to a wireless pairing process initiation, such as by pressing the pair button 18 on the medical acquisition device 10. Also in step 202, a match count is started at zero. In step 204, the medical acquisition device 10 produces a key pattern 42 comprised of one or more selected individual indicators 40, whereby the key pattern 42 represents a digit from the authentication key to be entered into the host device 60. In step 206, the host device 60 provides selectable targets 80 corresponding to the individual indicators 40 and further receives selections to form an overall entry pattern 82 intended to correspond to the key pattern 42. The individual pairing digit corresponding to each entry pattern 82 entered on the host device 60 is the transmitted to the medical acquisition device 10.

The control system 14 within the acquisition device 10 then determines whether the entry pattern 82 entered into the host device 60 matches the key pattern 42 provided with the medical acquisition device 10. If no match is confirmed in step 210, it is then determined whether there is any time remaining in step 212, as discussed above with respect to the standard time out of thirty seconds. If there is no time remaining, the process times out in step 214. If instead there is time remaining, the process continues with retrying a selection of targets 80 for the entry pattern 82, for example after clearing out the erroneous previous selection.

If instead it is confirmed that the entry pattern 82 entered into the host device 60 matches the key pattern 42 of the medical acquisition device 10 in step 210, the prior match count is increased by one in step 216, and is then determined in step 218 whether all 6 matches corresponding to the authentication key have been completed. If so, the authentication key has been successfully entered in its entirety and the wireless pairing process is completed in step 220. If instead not all six matches have been completed, the process continues to step 222 to determine whether time remains before the time out. If not, the process is timed out once again at step 214. However, if time is remaining in step 222 and not all six matches have been completed as determined at step 218, the process continues whereby the next sequential key pattern 42 is provided with a medical acquisition device 10, for subsequent entry into the host device 60 as an entry pattern 82.

Additional information is now provided for an exemplary control system 100, which is shown in FIG. 5. Certain aspects of the present disclosure are described or depicted as functional and/or logical block components or processing steps, which may be performed by any number of hardware, software, and/or firmware components configured to perform the specified functions. For example, certain embodiments employ integrated circuit components, such as memory elements, digital signal processing elements, logic elements, look-up tables, or the like, configured to carry out a variety of functions under the control of one or more processors or other control devices. The connections between functional and logical block components are merely exemplary, which may be direct or indirect, and may follow alternate pathways.

The control system 100 may be a computing system that includes a processing system 110, memory system 120, and input/output (I/O) system 130 for communicating with other devices, such as input devices 99 and output devices 101, either of which may also or alternatively be stored in a cloud 102. The processing system 110 loads and executes an executable program 122 from the memory system 120, accesses data 124 stored within the memory system 120, and directs the system 1 to operate as described in further detail below.

The processing system 110 may be implemented as a single microprocessor or other circuitry, or be distributed across multiple processing devices or sub-systems that cooperate to execute the executable program 122 from the memory system 120. Non-limiting examples of the processing system include general purpose central processing units, application specific processors, and logic devices.

The memory system 120 may comprise any storage media readable by the processing system 110 and capable of storing the executable program 122 and/or data 124. The memory system 120 may be implemented as a single storage device, or be distributed across multiple storage devices or sub-systems that cooperate to store computer readable instructions, data structures, program modules, or other data. The memory system 120 may include volatile and/or non-volatile systems, and may include removable and/or non-removable media implemented in any method or technology for storage of information. The storage media may include non-transitory and/or transitory storage media, including random access memory, read only memory, magnetic discs, optical discs, flash memory, virtual memory, and non-virtual memory, magnetic storage devices, or any other medium which can be used to store information and be accessed by an instruction execution system, for example.

The inventor has identified that others have attempted to institute wireless pairing without the use of an alphanumeric display, but have not solved the problems defined above. In particular, these prior art solutions (for example, U.S. Pat. No. 7,831,207) do not fully integrate within conventional Bluetooth® procedures. In addition, no presently known solutions are capable of reliably performing in the context of a Bluetooth® procedure that includes a thirty second time limit for providing all six authentication key entries. In the disclosed methods of U.S. Pat. No. 7,831,207, a light on the device is caused to blink for the number of times corresponding to the digit to be displayed. For example, to display digit “1”, the light blinks a single time, twice for “2,” and the like. There is an inter-group pause between the blinks to enable the user to distinguish between these groupings of blinks, which must be a longer pause than an intra-group pause between each blink sequence to distinguish between different digits of the pairing key. The patent specifies an interval of 0.5 seconds as the intra-group pause between blinks, and an inter-group pause of three seconds before the start of the next blink sequence. Several variations of this scheme are outlined in the patent, such as audible beeps or vibrations.

However, the inventor has identified that the methods of U.S. Pat. No. 7,831,207 are problematic. First, there is no mechanism for indicating via blinks, audible beeps, or vibrations the number zero, which prevents these methods from being directly applied to an authentication key possible under standard Bluetooth® protocols (which can have one or more zeros, even 000000). There is also a high possibility of error, whereby an authentication code of 999999 would necessitate correctly counting 9×6=54 light blinks, audible beeps, or vibrations. If even one blink, beep, or vibration is missed, the entire pairing process will necessarily fail.

Moreover, the methods of U.S. Pat. No. 7,831,207 are infeasible, or more likely impossible, under the Bluetooth® standards of today. When U.S. Pat. No. 7,831,207 was filed in 2007, the SMP timeout requirement of thirty seconds had not yet been implemented. Using the example of the authentication key 999999, along with the exemplary pauses provided in the patent as described above, it would be impossible to enter the key within the thirty second limit. Specifically, the intra-group pauses would include nine flashes, beeps, or vibrations×0.5 seconds×six digits for a total of twenty-seven seconds. In addition, the process would require five inter-group pauses×three seconds for a total of fifteen seconds. Therefore, the method would take a minimum of forty-two seconds, which is beyond the maximum of thirty seconds. Moreover, this ignores any time for the user to process these flashes, beeps, or vibrations and successfully input them into the receiving device. In short, the methods of U.S. Pat. No. 7,831,207 are not viable for Bluetooth® pairing under the standards of today, necessitating the systems and methods presently disclosed.

The foregoing has principally focused on Bluetooth® pairing methods referred to as “passkey entry pairing” in which one device knows and displays a key, which the user has to enter into the other device. The presently disclosed system and methods are also applicable to other methods of Bluetooth® pairing, including another referred to as “numeric comparison”. In Numeric Comparison pairing methods, both devices know the pairing key and the user has to provide a YES/NO answer to each digit displayed on either device.

In the example of the pairing key being 123456, both devices simultaneously derive this key. The medical acquisition device 10 lights up the LED corresponding to digit 1. The host device 60 lights up the LED corresponding to digit 1 on its graphic. The user verifies that both devices agree on the first digit of the pairing key sequence. The user presses a button on the medical acquisition device 10 corresponding to “YES”. The medical acquisition device 10 moves on to display the next digit. The user presses a button/control on the graphic on the host device 60 that corresponds to YES. The host device 60 moves on to display the next digit. If six YES entries are made on each device, the pairing succeeds. If both devices do not agree on a pairing digit, the user selects NO on either one of the devices and the pairing process fails.

For this pairing method to be implemented, in addition to a way of representing the pairing digits one-by-one on either device, two buttons corresponding to YES/NO are needed on each device. The two buttons on the medical acquisition device 10 described above (pairing button 18 and operating button 20) can be repurposed to temporarily serve these functions during the pairing process, for example.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A method for wirelessly pairing a medical acquisition device and a host device, the method comprising:
requesting an authentication key associated with the medical acquisition device to be entered into the host device;
producing a key pattern on the medical acquisition device representative of the authentication key, wherein the key pattern is non-numeric;
providing a plurality of targets on the host device;
receiving selections of the plurality of targets to form an entry pattern on the host device; and
wirelessly pairing the medical acquisition device and the host device only when the entry pattern is determined to match the key pattern on the medical acquisition device.

2. The method according to claim 1, wherein each entry pattern includes the selection of at least one of the plurality of targets.

3. The method according to claim 1, wherein the key pattern is provided as multiple subpatterns, wherein the entry pattern is multiple subselections, and wherein the step of providing each of the multiple subspatterns is followed by a corresponding step of receiving one of the multiple subselections.

4. The method according to claim 3, wherein the multiple subpatterns is six subpatterns and the multiple subselections is six subselections.

5. The method according to claim 4, wherein the authentication key is a number having six digits, and wherein each of the six subpatterns corresponds to one of the six digits.

6. The method according to claim 4, wherein the key pattern is formed by selection among a plurality of individual indicators, wherein the plurality of individual indicators is at least four individual indicators.

7. The method according to claim 6, wherein the key pattern is one of at least ten unique patterns selectable by the at least four individual indicators.

8. The method according to claim 3, further comprising indicating with the host device how many of the multiple subselections have been received.

9. The method according to claim 3, wherein a time limit is provided for receiving the selections after the key pattern is provided, further comprising indicating with the host device a remaining time until the time limit is reached.

10. The method according to claim 9, further comprising indicating with the host device how many of the multiple subselections have been received.

11. The method according to claim 1, wherein the key pattern is formed by selection among a plurality of individual indicators, wherein the plurality of individual indicators are arranged in a particular orientation, and wherein the plurality of targets on the host are arranged to correspond to the particular orientation of the plurality of individual indicators.

12. The method according to claim 11, wherein the plurality of individual indicators is ten individual indicators.

13. The method according to claim 11, wherein each key pattern is formed by the selection of only one of the plurality of individual indicators.

14. The method according to claim 1, wherein the key pattern is produced by selective illumination of lights.

15. The method according to claim 14, wherein the lights are LEDs and the medical acquisition device provides ECG data.

16. The method according to claim 15, wherein the lights comprise ten lights each corresponding to one of ten ECG lead placement positions.

17. The method according to claim 1, wherein the selections for the entry pattern are receivable via a user input device.

18. The method according to claim 17, wherein the user input device is a mouse.

19. A method for wirelessly pairing a medical acquisition device and a host device within a time limit, the method comprising:
providing a plurality of targets on the host device;
requesting the medical acquisition device to sequentially produce six key patterns, each of the six key patterns being non-numeric and comprised of selections among a plurality of individual indicators;
sequentially receiving on the acquisition device, one after each of the six key patterns is produced on the host device, six entry patterns each comprised of selections among the plurality of targets;
indicating how many of the six entry patterns have been entered at the host device;
indicating a remaining time until the time limit is reached; and
wirelessly pairing the medical acquisition device and the host device only when the sequence of six entry patterns is determined to match the sequence of six key patterns on the medical acquisition device before the time limit is reached.

20. A system for wirelessly pairing a medical acquisition device and a host device, the system comprising:
a plurality of LEDs operative coupled to the medical acquisition device, wherein each of the plurality of LEDs is selectively illuminated to collectively form at least six key patterns, wherein the at least six key patterns are non-numeric;
a display device operatively coupled to the host device, wherein the display device displays a plurality of targets thereon, wherein each of the plurality of targets is selectable;
an input device operatively coupled to the host device, wherein the input device is operable to select among the plurality of targets to collectively form entry patterns; and
a computer system that compares the at least six key patterns to the entry patterns corresponding thereto;
wherein the medical acquisition device and the host device wireless pair only when the at least six key patterns are determined to match the entry patterns corresponding thereto.
